(19) United States
(12) Patent Application Publication
(10) Pub. No.: US 2008/0103310 A1
(43) Pub. Date: May 1, 2008

(54) Preparation of 1H-imidazo[4,5-c]quinolin-4-amines via 1H-imidazo[4, 5-c]quinolin-4-phtalimide intermediates

(76) Inventors: Janos Hajko, Debrecen (HU); Csaba Szabo, Debrecen (HU); Piroska Kovacs, Debrecen (HU)

Correspondence Address: KENYON & KENYON LLP, ONE BROADWAY, NEW YORK, NY, 10004, US

(21) Appl. No.: 11/974,947

(22) Filed: Oct. 16, 2007

Related U.S. Application Data
(60) Provisional application No. 60/852,153, filed on Oct. 16, 2006.
(60) Provisional application No. 60/899,974, filed on Feb. 6, 2007.
(60) Provisional application No. 60/920,349, filed on Mar. 26, 2007.
(60) Provisional application No. 60/899,874, filed on Feb. 7, 2007.

(51) Int. Cl. C07D 471/04

(52) U.S. Cl. 546/82

(57) ABSTRACT

The present invention provides process for the preparation of 4-amino-1H-imidazo[4,5-c]quinolines comprising the step of reacting a 1H-imidazo[4,5-c]quinolin-4-phthalimide with an amine selected from the group consisting of: alkylamine, aralkylamine, alkyldiamine, and aralkyldiamine.

### CROSS REFERENCE TO RELATED APPLICATIONS

This application claims the benefit of U.S. Provisional Patent Application No. 60/852,153, filed Oct. 16, 2006; U.S. Provisional Patent Application No. 60/899,974, filed Feb. 6, 2007; and U.S. Provisional Patent Application No. 60/920,349, filed Mar. 26, 2007; the contents of which are incorporated herein by reference.

### FIELD OF THE INVENTION

The present invention relates to a process for the synthesis of 1H-imidazo[4,5-c]quinolin-4-amines. More particularly, the present invention relates to a process for the preparation Imiquimod via 1H-imidazo[4,5-c]quinolin-4-phthalimide.

### BACKGROUND OF THE INVENTION

Imiquimod, 4-amino-1-isobutyl-1H-imidazo[4,5-c]quinoline of the following structure, 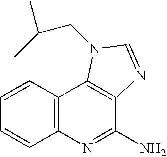
is an immune response modifier, useful for treating viral infections, such as genital warts. Imiquimod developed by 3M Pharmaceuticals is marketed as a cream, under the trade name ALDARA®.

One of the routes to prepare Imiquimod is disclosed in WO patent application No. 2004/009593, and is illustrated by the following scheme: 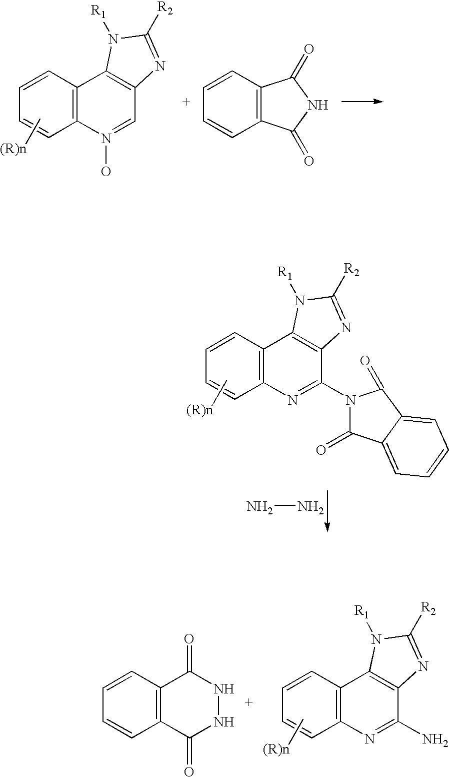
wherein a phthalimide group is introduced in the first stage, and then removed using hydrazine hydrate in a solvent mixture of water and methanol.

Carbohydrate Research, 1993, 243, 139-164, discloses the removal of a phthalimide group from trisaccharides, which represent very different chemical structures from the 1H-imidazo[4,5-c]quinolines disclosed herein. This method uses a large excess of an amine instead of hydrazine. The method disclosed in this publication uses 400-600 equivalents of amine for the removal of one phthalimido group.

### SUMMARY OF THE INVENTION

In one embodiment, the present invention provides a process for preparing a 4-amino-1H-imidazo[4,5-c]quinoline of formula I

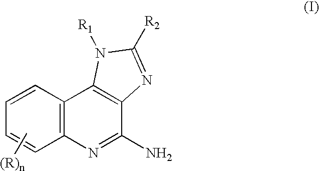

from a 1H-imidazo[4,5-c]quinolin-4-phthalimide of formula II,

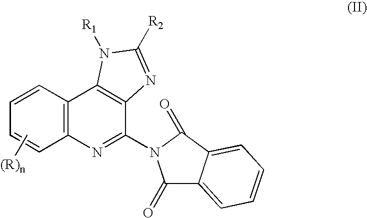

comprising reacting the 1H-imidazo[4,5-c]quinolin-4-phthalimide of formula II and an amine selected from the group consisting of: alkylamine, aralkylamine, alkyldiamine, and aralkyldiamine; wherein R₁ and R₂ are independently selected from the group consisting of: hydrogen, a straight or branched chain alkyl, aromatic hydrocarbon, and phenyl substituted aromatic hydrocarbon, wherein R is selected from the group consisting of: alkoxy, alkyl, and halogen; and n is an integer from 0 to 2, with the proviso that if n is 2, then said groups together contain no more than 6 carbon atoms.

In the above embodiments:

Preferably, the alkylamine is a C₁₋₆ alkylamine.

Preferably, the alkyldiamine is a C₁₋₆ alkyldiamine.

Preferably, the aralkyldiamine is C₆₋₈ aralkyldiamine.

Preferably, the alkyl is C₁₋₁₀ straight or branched chain alkyl; more preferably C₁₋₈ straight or branched chain alkyl; even more preferably C₁₋₇ straight or branched chain alkyl.

Preferably, the alkoxy is C₁₋₄ alkoxy.

Preferably, the halogen is F, Cl, Br, or I; more preferably F.

Preferably, the aromatic hydrocarbon is C₆₋₁₂ aromatic hydrocarbon.

In another embodiment, the present invention provides a process for preparing a 4-amino-1H-imidazo[4,5-c]quinoline of formula I

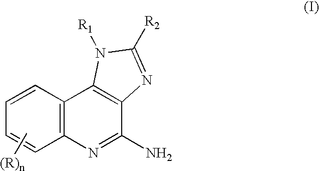

from a 1H-imidazo[4,5-c]quinolin-N-oxide of formula III

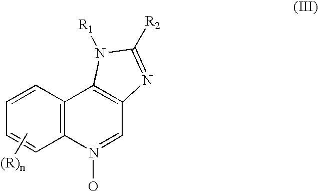

comprising reacting the 1H-imidazo[4,5-c]quinolin-N-oxide of formula III, with phthalimide to obtain a 1H-imidazo[4,5-c]quinolin-4-phthalimide of formula II, and reacting the 1H-imidazo[4,5-c]quinolin-4-phthalimide of formula II and an amine selected from the group consisting of: alkylamine, aralkylamine, alkyldiamine, and aralkyldiamine; wherein R₁ and R₂ are independently selected from the group consisting of: hydrogen, a straight or branched chain alkyl, aromatic hydrocarbon, and phenyl substituted aromatic hydrocarbon; wherein R is selected from the group consisting of: alkoxy, alkyl, and halogen; and n is an integer from 0 to 2, with the proviso that if n is 2, then said groups together contain no more than 6 carbon atoms.

### DETAILED DESCRIPTION OF THE INVENTION

The present invention is directed to a process for preparing 1H-imidazo[4,5-c]quinolin-4-amines of formula I, in particular 4-amino-1-isobutyl-1H-imidazo[4,5-c]quinoline, Imiquimod, using a non-carcinogenic, cheap and efficient agent for removing the phthalimide group.

The process may be done according to the following scheme:

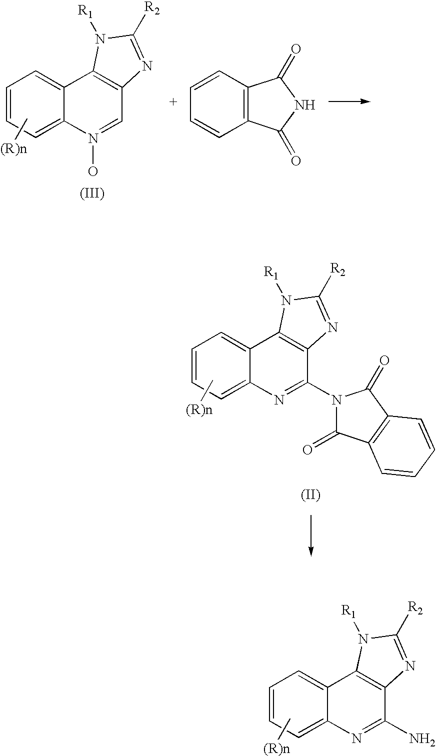

wherein R₁ and R₂ are independently selected from the group consisting of: hydrogen, a straight or branched chain alkyl, aromatic hydrocarbon, and phenyl substituted aromatic hydrocarbon wherein R is selected from the group consisting of: alkoxy, alkyl, and halogen; and n is an integer from 0 to 2, with the proviso that if n is 2, then said groups together contain no more than 6 carbon atoms.

Preferably for R₁ and R₂ the straight or branched chain alkyl is C₁₋₁₀ straight or branched chain alkyl; more preferably C₁₋₈ straight or branched chain alkyl; even more preferably C₁₋₇ straight or branched chain alkyl; and most preferably C₁₋₄ straight or branched chain alkyl. Preferably, the C₁₋₄ straight or branched chain alkyl is methyl, ethyl, n-propyl, isopropyl, n-butyl, isobutyl, or tert-butyl; more preferably isobutyl. Preferably, the aromatic hydrocarbon is C₆₋₁₂ aromatic hydrocarbon; more preferably C₆₋₈ aromatic hydrocarbon; most preferably phenyl, tolyl, or xylyl; most preferably phenyl. Preferably, the phenyl substituted aromatic hydrocarbon contains one or two substituents on the benzene ring. Preferably, the substituents for the group R are selected from the group consisting of: a C₁₋₄ alkyl group C₁₋₄ alkoxy group, and halogen with the proviso that when the benzene ring of the compound of Formulae (I), (II) or (III) is substituted by two groups, the total number of carbon atoms on the substituents is no more than 6. Preferably, the C₁₋₄ alkyl group is methyl, ethyl, n-propyl, isopropyl, n-butyl, isobutyl, or tert-butyl; more preferably methyl. Preferably, the C₁₋₄ alkoxy group is methoxy, ethoxy, n-propoxy, isopropoxy, n-butoxy, isobutoxy, or tert-butoxy; more preferably methoxy. Preferably, the halogen is F, Cl, Br, or I; more preferably F. Most preferably, R₁ is isobutyl. Most preferably, R₂ is hydrogen. Most preferably, R is hydrogen.

Preferably, when R₁ is isobutyl, R₂ is hydrogen, and n is 0, said compound of formula I refers to 4-amino-1-isobutyl-1H-imidazo[4,5-c]quinoline (referred to as Imiquimod) of the following formula, 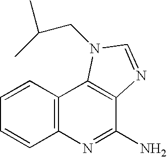
said formula II corresponds to 1-isobutyl-1H-imidazo[4,5-c]quinolin-4-phthalimide of the following formula, 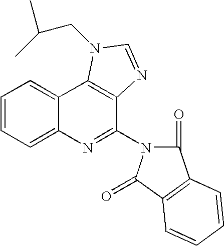
and said compound of formula III corresponds to 1-isobutyl-1H-imidazo[4,5-c]quinolin-N-oxide of the following formula.

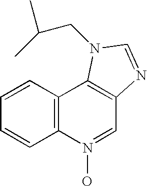

1H-imidazo[4,5-c]quinolin-N-oxide of formula III can be prepared, for example, according to the process disclosed in WO 2004/009593.

The compound of formula II can be obtained, for example, according to the process disclosed in WO patent application 2004/009593 or in Example 1 herein. The process comprises reacting the 1H-imidazo[4,5-c]quinolin-N-oxide of formula III

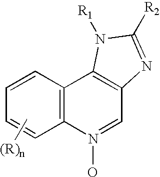

with phthalimide, wherein R₁, R₂, R and n are as described above.

The compound of formula II may react with the amine according to the above scheme without being recovered prior to the reaction, i.e., one-pot reaction. Preferably, the compound of formula II is recovered prior to reacting with the amine.

1H-imidazo[4,5-c]quinolin-4-phthalimide of formula II,

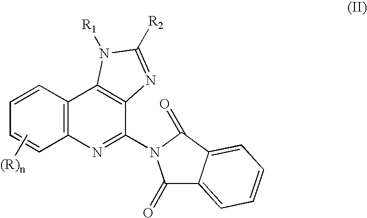

is then converted to 4-amino-1H-imidazo[4,5-c]quinoline of formula I

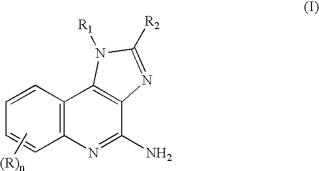

by a process comprising reacting 1H-imidazo[4,5-c]quinolin-4-phthalimide of formula II and an amine selected from the group consisting of: alkylamine, aralkylamine, alkyldiamine, and aralkyldiamine; wherein R₁ and R₂ are independently selected from the group consisting of: hydrogen, a straight or branched chain alkyl, aromatic hydrocarbon, and substituted aromatic hydrocarbon; wherein R is selected from the group consisting of: alkoxy, alkyl, and halogen; and n is an integer from 0 to 2, with the proviso that if n is 2, then said groups together contain no more than 6 carbon atoms.

Preferably, R, R₁, R₂ and n are as described above.

The reaction between the compound of formula II and the amine is carried out in a solvent selected from the group consisting of: water; alcohol; linear, branched, and cyclic ether; aliphatic hydrocarbon, aromatic hydrocarbon; nitroalkane; alkylcyanide; and mixtures thereof.

Preferably, the alcohol is C₁₋₄ alcohol, methanol, ethanol, n-propanol, isopropanol, n-butanol, isobutanol, or tert-butanol; more preferably methanol. Preferably, the linear, branched or cyclic ether is linear, branched, or cyclic C₂₋₈ ether. Preferably, the linear, branched, or cyclic C₂₋₈ ether is diethyl ether, diisopropyl ether, or tetrahydrofuran; more preferably tetrahydrofuran. Preferably, the aliphatic hydrocarbon is C₅₋₈ aliphatic hydrocarbon; more preferably n-pentane, n-hexane, cyclohexane, n-heptane, or n-octane; most preferably n-hexane. Preferably, the nitroalkane is C₁₄ nitroalkane; more preferably nitromethane, nitroethane, nitropropane, or nitrobutane; most preferably nitromethane. Preferably, the alkylcyanide is C₁₋₄ alkylcyanide; more preferably acetonitrile, propionitrile, or butyronitrile; most preferably acetonitrile. Preferably, the aromatic hydrocarbon is a C₆₋₈ aromatic hydrocarbon, more preferably benzene, toluene or xylene, most preferably toluene. The most preferred solvent is water.

Preferably, the alkylamine is C₁₋₆ alkylamine. Preferably, the C₁₋₆ alkylamine is primary amine; more preferably methylamine, ethylamine, propylamine, butylamine, pentylamine, or hexylamine; most preferably methylamine. Preferably, the aralkylamine is C₆₋₈ aralkylamine; more preferably benzylamine or 4-methylbenzylamine; most preferably benzylamine. Preferably, the alkyldiamine is C₁₋₆ alkyldiamine; more preferably ethylenediamine (1,2-diaminoethane), diaminopropane, diaminobutane, diaminopentane, or diaminohexane; most preferably ethylenediamine. The alkyldiamines are preferably terminal amines—i.e. amines having general formula: H₂N(CH₂)ₘNH₂. Preferably, the aralkyldiamine is C₆₋₈ aralkyldiamine; more preferably xylylenediamine or aminoethylaniline most preferably xylylenediamine. The most preferred amine is ethylenediamine.

Preferably, the amine is present in an amount of about 1 to about 10 mole equivalents per mole equivalent of compound of formula II; more preferably about 1.5 to about 5 mole equivalents; and most preferably about 1.5 to about 2 mole equivalents per mole equivalent of compound of formula II. In one embodiment, the molar ratio is about 1.5 to about 2.5 mole equivalents.

Preferably, the amine is added to a suspension or solution of the compound of formula II in the solvent, depending on the kind of the solvent. Preferably, the addition is done drop-wise. Preferably, the addition is done over a period of about 1 minute to about 60 minutes; more preferably for about 5 to about 30 minutes. Preferably, the addition is done at a temperature of about 40° C. to about 90° C.; more preferably at about 60° C. to about 80° C.

Preferably, a mixture is obtained after the addition. Preferably, the mixture is heated to a temperature of about 40° C. to about 100° C.; more preferably about 90° C. to about 95° C. Preferably, the heating is done for about 2 to about 12 hours; more preferably for about 3 to about 6 hours. The conversion of the compound of Formula II to Formula I can be monitored by TLC, preferably using a mixture of dichloromethane and methanol in a ratio of 8 to 2 as an eluent.

The reaction of compound of formula II and the amine may further comprise a recovery process. The recovery may be done by any method known to the skilled artisan. Preferably, the recovery process may include cooling the heated mixture; adding an alcoholic solvent such as methanol, ethanol, or propanol; maintaining the mixture at a temperature of about 50° C. to about 90° C.; more preferably about 60° C. to about 65° C.; cooling the mixture; and filtering the product. Preferably, the heated mixture is cooled to a temperature of about 65° C. to about 20° C.; more preferably to about 60° C. to about 40° C. Preferably, the mixture is maintained for about 5 to about 60 minutes; more preferably for about 10 to about 30 minutes. Preferably, the cooling step prior to filtering the product compound of formula I is done to a temperature of about 45° C. to about 5° C.; more preferably to about 20° C. to about 25° C.

The quality and yield of the crude compound of formula I, in particular of Imiquimod are influenced by the process of removing the phthalimide group. The recovered Imiquimod may be obtained by the above process in a purity of about 99.0% to about 99.5% by HPLC and in yields of about 75% to about 85% by weight. Preferably, it may contain less than about 0.1%, preferably less than about 0.05-0.15%, and more preferably less than about 0.08 to about 0.12% area by HPLC of Imiquimod-OH of the following formula:

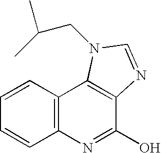

More preferably, it may contain about 0.1% area by HPLC to about 0.01% area by HPLC of the above Imiquimod-OH.

To increase the level of purity, the compound of formula I may be purified by reacting it with an acid to obtain the salt of the compound of formula I, 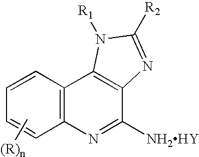
and reacting with a base, to obtain back the compound of formula I, as described, for example, in Example 7.

The present invention is illustrated in further details with reference to the following non limiting examples.

### EXAMPLES

### Example 1

### Preparation of 1-isobutyl-1H-imidazo[4.5-c]quinolin-4-phthalimide

To a stirred suspension of 1-isobutyl-1H-imidazo[4.5-c]quinolin-N-oxide (700 g, 2900 mmol) in ethyl acetate (3500 ml), tributylamine (1750 ml, 2.5 equiv) and phthalimide (490 g, 1.2 equiv) were added. The suspension was cooled to 0-5° C. and then, within 1.5-2 hours, benzoyl chloride (480 ml, 1.4 equiv) was added portion-wise (temperature was kept between 0-5° C.). The suspension was heated to 20-25° C. and stirred for an additional 2 hours (conversion was monitored by TLC using 10:1 DCM-MeOH as an eluent). The reaction mixture was filtered and the cake was washed with ethyl acetate (4×500 ml) and then with methanol (2×500 ml). The wet solid was suspended in methanol (6 L) and stirred for 5 hours at 40-45° C., and then cooled to 20-25° C. The suspension was filtered and the cake was washed with methanol (3×600 ml). The wet material was dried under vacuum at 50° C. for 12 hours to obtain 1-isobutyl-1H-imidazo[4.5-c]quinolin-4-phthalimide (910 g, 84.6%).

HPLC: 98.63 a % of 1-isobutyl-1H-imidazo[4.5-c]quinolin-4-phthalimide and 0.19 a % of Imiquimod-OH

### Example 2

### Preparation of Imiquimod

To a stirred suspension of 1-alkyl-1H-imidazo[4.5-c]quinolin-N-oxide (290 mmol) in ethyl acetate (350 ml) is added tributylamine (175 ml, 2.5 equiv) and phthalimide (49 g, 1.2 equiv). The suspension is cooled to 0-5° C. and then within 1.5-2 hours benzoyl chloride (48 ml, 1.4 equiv) is added portion-wise (temperature is kept between 0-5° C.). The suspension is heated to 20-25° C. and stirred for additional 2 hours (conversion is monitored by TLC using 10:1 DCM-MeOH as an eluent). The reaction mixture is filtered and the cake is washed with ethyl acetate (4×100 ml) and then with methanol (2×100 ml). The wet solid is suspended in methanol (0.6 L) and stirred for 5 hours at 40-45° C., and then cooled to 20-25° C. The suspension is filtered and the cake was washed with methanol (2×120 ml) and water (2×120 ml). The wet material is suspended in water (350 ml) and ethylenediamine-(32 ml, 2 equiv) was added drop-wise at 70° C. The mixture is heated to 90-95° C. and stirred for 4 hours (conversion is monitored by TLC using 8:2 DCM-MeOH as an eluent).

After cooling to 60° C., methanol is added (870 ml) and the reaction mixture is stirred at reflux temperature for 15 min. After cooling to 20-25° C. the mixture is filtered and the cake is washed with aqueous methanol (MeOH:H₂O=3.5:1 v/v, 2×120 ml) and water (2×120 ml). The wet solid material is suspended in water (870 ml) and the stirred suspension is treated with 37% HCl (22 ml, 1.1 equiv), then heated to 90-93° C. and this temperature is maintained for 30 min. The hot solution is treated with sodium dithionite (Na₂S₂O₄, 0.6 g, ca 1%) and charcoal (3 g, ca 5%). After 30 min the mixture is filtered and the cake is washed with water (2×60 ml). The filtrate is cooled to 70-75° C. and the pH is adjusted to 11.4-11.6 by the addition of aqueous 30% NaOH (ca 40 ml). The stirred mixture is cooled to 20-25° C. and after 1 hour the solid material is filtered off. The cake is washed with water (3×90 ml) and the wet solid is suspended in a mixture of water (265 ml) and n-butanol (117 ml). The stirred suspension is treated with 37% HCl (21 ml, 1.1 equiv) and then heated to 60-65° C. until complete dissolution occurred. The solution is cooled to 20-25° C. and the precipitated hydrochloride salt is filtered and then washed with n-butanol (53 ml). The wet hydrochloride salt is dissolved in water (580 ml) at 85-90° C. and the solution is filtered and the filtered solid is washed with hot water (27 ml). The filtrate is treated with sodium dithionite (Na₂S₂O₄, 0.1 g, ca 0.2%), cooled to 70-75° C., and the pH is adjusted to 9.6-9.8 by the addition of aqueous 30% NaOH (ca 20 ml). The stirred mixture is cooled to 20-25° C. and the solid material is filtered off. The cake is washed with water (4×80 ml) and methanol (2×50 ml), then dried under vacuum at 50° C. for 7-8 hours to give crystallized 1-alkyl-4-amino-1H-imidazo[4,5-c]quinoline (60-65%).

### Example 3

### Preparation of Crude Imiquimod

To a stirred suspension of 1-isobutyl-1H-imidazo[4.5-c]quinolin-4-phthalimide (740 g, 2 mol) in water (3000 ml), was added drop-wise ethylenediamine (270 ml, 2 equiv) at 70° C. The mixture was heated to 90-95° C. and stirred for 4 hours (conversion was monitored by TLC using 8:2 DCM-MeOH as an eluent).

After cooling to 60° C., methanol was added (7400 ml) and the reaction mixture was stirred at reflux temperature for 15 min. After cooling to 20-25° C., the mixture was filtered and the cake was washed with aqueous methanol (MeOH:H₂O=3.5:1 v/v, 3×620 ml). The wet solid material was dried under vacuum at 50° C. for 7-8 hours to obtain crude Imiquimod (441 g, 91.8%). HPLC: 99.40 a % of Imiquimod and 0.09 a % of Imiquimod-OH

### Example 4

### Purification of Crude Imiquimod

The dried crude Imiquimod (440 g) was suspended in water (7400 ml) and the stirred suspension was treated with 37% HCl (180 ml) and then heated to 90-93° C., and this temperature was maintained for 30 min. The hot solution was treated with sodium dithionite (Na₂S₂O₄, 5 g, ca 1%) and charcoal (24 g, ca 5%). After 30 min, the mixture was filtered and the cake was washed with water (2×500 ml). The filtrate was cooled to 70-75° C. and the pH was adjusted to 11.4-11.6 by the addition of aqueous 30% NaOH. The stirred mixture was cooled to 20-25° C. and after 1 hour the solid material was filtered off. The cake was washed with water (3×500 ml) and methanol (90 ml), then dried under vacuum at 50° C. for 7-8 hours to give purified Imiquimod (421 g, 95.7%). HPLC: 99.77 a % of Imiquimod and 0.07 a % of Imiquimod-OH

### Example 5

### Crystallization of Purified Imiquimod

The dried purified Imiquimod (400 g, 1.66 mol) was suspended in a mixture of water (2000 ml) and n-butanol (900 ml) and the stirred suspension was treated with 37% HCl (150 ml, 1.1 equiv), and then heated to 60-65° C. until complete dissolution occurred. The solution was cooled to 20-25° C. and the precipitated Imiquimod hydrochloride was filtered and then washed with n-butanol (400 ml). The wet hydrochloride salt* was dissolved in water (4500 ml) at 85-90° C., the solution was filtered, and the filtered solid was washed with hot water (200 ml). The filtrate was treated with sodium dithionite (Na₂S₂O₄, 0.9 g, ca 0.2%), cooled to 70-75° C., and the pH was adjusted to 9.6-9.8 by the addition of aqueous 30% NaOH (ca 160 ml). The stirred mixture was cooled to 20-25° C. and the solid material was filtered off. The cake was washed with water (3×200 ml) and methanol (2×200 ml) and then dried under vacuum at 50° C. for 7-8 hours to give crystallized Imiquimod (351 g, 87.8%) HPLC: 99.97 a % of Imiquimod and 0.03 a % of Imiquimod-OH
*If necessary wet hydrochloride salt can be dried at 50° C.

### Example 6

### Preparation of 1H-imidazo[4,5-c]quinolin-N-oxide according to WO 2004/009593

The oxidation of 1-isobutyl-1H-imidazo[4,5-c]quinoline (which may be produced as in Example 3 of WO 2004/009593) is carried out in toluene at 40-45° C. using peracetic acid as oxidant to produce 1-isobutyl-1H-imidazo[4,5-c]quinoline N-oxide. The product is isolated by filtration after addition of a sodium sulfate solution and ammonium hydroxide.

### Example 7

### Purification of Imiquimod according to WO 2004/009593

53.55 ml water, 23.62 ml butyl alcohol, 10.57 crude Imiquimod and 4.77 g of 37% HCl are loaded into a 100 ml reactor. The mixture is heated to 55-60° C. to obtain a solution. The solution is cooled to room temperature and a white crystal precipitates. The solid is filtered and washed 2 times with 5 ml butyl alcohol. 13.63 g of wet Imiquimod hydrochloride is obtained.

HPLC analysis shows that there is 99.89% Imiquimod and 0.01% phthalhydrazide. 120 ml water and 13.63 g of wet Imiquimod hydrochloride are loaded into a 250 ml reactor and heated to 85-90° C. The hot solution is filtered and the cake is washed with 5 ml of hot water. Then 0.024 g of Na₂S₂O₄ is added. The colorless solution is cooled to 70-75° C. and 5.3 g of 30% NaOH is added to provide a pH of 9.7, at which point a solid precipitates. The suspension is cooled to 20° C. and filtered. The cake is washed 3 times with 5 ml water and twice with 5 ml methanol. During the washes no chloride was detected by silver nitrate. The solid is dried under vacuum at 50° C. for 8 hours. 8.98 g of Imiquimod (off-white color) is obtained. HPLC shows the purity to be 99.94% and the yield to be 63.3% based on the starting material (1-isobutyl-1H-imidazo[4,5-c]quinoline N-oxide).

What is claimed is:

1. A process for preparing a 4-amino-1H-imidazo[4,5-c]quinoline of formula I 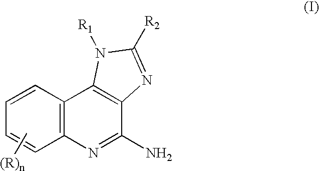
comprising reacting the 1H-imidazo[4,5-c]quinolin-4-phthalimide of formula II 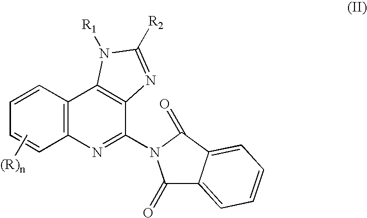
and an amine selected from the group consisting of: alkylamine, aralkylamine, alkyldiamine, and aralkyldiamine; wherein R₁ and R₂ are independently selected from the group consisting of: hydrogen, a straight or branched chain alkyl, aromatic hydrocarbon, and phenyl substituted aromatic hydrocarbon; wherein R is selected from the group consisting of: alkoxy, alkyl, and halogen; and n is an integer from 0 to 2, with the proviso that if n is 2, then said groups together contain no more than 6 carbon atoms.

2. The process of claim 1, wherein R₁ and R₂ are independently selected from the group consisting of: hydrogen, C₁₋₁₀ straight or branched chain alkyl, a C₆₋₁₂ aromatic hydrocarbon, a C₁₋₄ alkyl phenyl substituted aromatic hydrocarbon, C₁₋₄ alkoxy group phenyl substituted aromatic hydrocarbon, and halogen phenyl substituted aromatic hydrocarbon.

3. The process of claim 2, wherein R₁ and R₂ are independently selected from the group consisting of: hydrogen, methyl, ethyl, n-propyl, isopropyl, n-butyl, isobutyl, tert-butyl; phenyl, tolyl, xylyl, methyl phenyl substituted aromatic hydrocarbon, ethyl phenyl substituted aromatic hydrocarbon, n-propyl phenyl substituted aromatic hydrocarbon, isopropyl phenyl substituted aromatic hydrocarbon, n-butyl phenyl substituted aromatic hydrocarbon, isobutyl phenyl substituted aromatic hydrocarbon, tert-butyl phenyl substituted aromatic hydrocarbon; methoxy phenyl substituted aromatic hydrocarbon, ethoxy phenyl substituted aromatic hydrocarbon, n-propoxy phenyl substituted aromatic hydrocarbon, isopropoxy phenyl substituted aromatic hydrocarbon, n-butoxy phenyl substituted aromatic hydrocarbon, isobutoxy phenyl substituted aromatic hydrocarbon, tert-butoxy phenyl substituted aromatic hydrocarbon; F phenyl substituted aromatic hydrocarbon, Cl phenyl substituted aromatic hydrocarbon, Br phenyl substituted aromatic hydrocarbon, and I phenyl substituted aromatic hydrocarbon.

4. The process of claim 3, wherein R₂ is hydrogen.

5. The process of claim 4, wherein R₁ is hydrogen, methyl, ethyl, n-propyl, isopropyl, n-butyl, isobutyl, tert-butyl; phenyl, tolyl, xylyl, and preferably isobutyl.

6. The process of claim 1, wherein R is H, R₁ is isobutyl, R₂ is hydrogen, and n is 0.

7. The process of claim 1, wherein the reaction of the compound of formula II and the amine is carried out in a solvent selected from the group consisting of: water; alcohol; linear, branched, and cyclic ether; aliphatic hydrocarbon, aromatic hydrocarbon, nitroalkane; alkylcyanide; and mixtures thereof.

8. The process of claim 7, wherein the solvent is selected from the group consisting of: C₁₋₄ alcohol, linear, branched, or cyclic C₂₋₈ ether, C₅₋₈ aliphatic hydrocarbon, C₁₋₄ nitroalkane, C₁₋₄ alkylcyanide, C₆₋₈ aromatic hydrocarbon, water and mixtures thereof.

9. The process of claim 8, wherein the solvent is a C₆₋₈ aromatic hydrocarbon.

10. The process of claim 9, wherein the solvent is benzene, toluene or xylene.

11. The process of claim 10, wherein the solvent is toluene.

12. The process of claim 8, wherein the solvent is selected from a group consisting of: methanol, ethanol, n-propanol, isopropanol, n-butanol, isobutanol, tert-butanol, diethyl ether, diisopropyl ether, tetrahydrofuran, n-pentane, n-hexane, cyclohexane, n-heptane, n-octane, nitromethane, nitroethane, nitropropane, nitrobutane, acetonitrile, propionitrile, butyronitrile, benzene, toluene or xylene, water and mixtures thereof.

13. The process of claim 8, wherein the solvent is water.

14. The process of claim 1, wherein the alkylamine is a C₁₋₆ alkylamine; the aralkylamine is a C₆₋₈ aralkylamine; the alkyldiamine is a C₁₋₆ alkyldiamine; the aralkyldiamine is a C₆₋₈ aralkyldiamine.

15. The process of claim 14 wherein the C₁₋₆ alkylamine is methylamine, ethylamine, propylamine, butylamine, pentylamine, or hexylamine; the C₆₋₈ aralkylamine is benzylamine or 4-methylbenzylamine; the C₁₋₆ alkyldiamine is ethylenediamine (1,2-diaminoethane), diaminopropane, diaminobutane, diaminopentane, or diaminohexane; and the C₆₋₈ aralkyldiamine is xylylenediamine or aminoethylaniline.

16. The process of claim 1, wherein the amine is ethylenediamine.

17. The process of claim 1, wherein the amine is present in an amount of about 1 to about 10 mole equivalents per mole equivalent of compound of formula II.

18. The process of claim 17, wherein the amine is present in an amount of about 1.5 to about 5 mole equivalents per mole equivalent of compound of formula II.

19. The process of claim 18, wherein the amine is present in an amount of about 1.5 to about 2.5 mole equivalents per mole equivalent of compound of formula II.

20. The process of claim 1, wherein the amine is added to a suspension or a solution of the compound of formula II in the solvent, providing a mixture.

21. The process of claim 20, wherein the mixture is heated to a temperature of about 40° C. to about 100° C.

22. The process of claim 1, further comprising recovery of the compound of formula I.

23. The process of claim 1, wherein the compound of formula II is obtained by a process comprising reacting a 1H-imidazo[4,5-c]quinolin-N-oxide of formula III 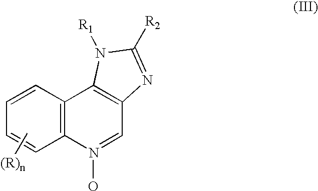
with phthalimide.

24. The process of claim 23, wherein the obtained compound of Formula II isn't isolated prior to ints conversion to the compound of formula I.
